(19) United States
(12) Patent Application Publication
(10) Pub. No.: US 2014/0275861 A1
(43) Pub. Date: Sep. 18, 2014

(54) AMBULATORY SENSING SYSTEM AND ASSOCIATED METHODS

(71) Applicant: CardioMEMS, Inc., Atlanta, GA (US)

(72) Inventors: Jason Mark Kroh, Atlanta, GA (US); Tamera Lee Scholz, Atlanta, GA (US)

(73) Assignee: CardioMEMS, Inc., Atlanta, GA (US)

(21) Appl. No.: 14/215,818

(22) Filed: Mar. 17, 2014

Related U.S. Application Data
(60) Provisional application No. 61/794,412, filed on Mar. 15, 2013.

(51) Int. Cl. A61B 5/00; G08C 17/02

(52) U.S. Cl. CPC A61B 5/002; G08C 17/02
USPC 600/302; 340/870.01

(57) ABSTRACT

A method for monitoring data in a clinical environment comprises receiving information indicative of a number of ambulatory sensing systems in a measurement environment comprising a plurality of ambulatory sensing systems. The method also comprises establishing a communication scheme associated with the measurement environment based on the number of ambulatory sensing systems in the measurement environment. The method further comprises providing, to each ambulatory sensing system, information indicative of the established communication scheme. The method also comprises configuring, in a central data module associated with the measurement environment, parameters for communicating with each respective ambulatory sensing system in accordance with the established control scheme.

### CROSS-REFERENCE TO RELATED APPLICATION

This application claims the benefit of U.S. Provisional Application No. 61/794,412, filed Mar. 15, 2013, which is incorporated herein by reference in its entirety.

### TECHNICAL FIELD

The present disclosure relates generally to monitoring of physiological parameters in a clinical environment and, more particularly, to systems and methods for effectively and remotely monitoring physiological parameters of a plurality of mobile subjects.

### BACKGROUND

Implantable LC pressure sensors are used in a variety of applications for measuring physiological parameters of a patient. However, multiple sensors in a single host may only be reliably interrogated if they are placed sufficiently far apart from one another such that the external electronics are able detect and independently acquire the signal from the sensor of interest without any parasitic effects caused by other sensors. Additionally, conventional sensors are limited in their ability to conveniently or reliably measure multiple subjects implanted with wireless passive sensors residing in a common area via a single interrogation system. Thus, a need exists for a sensing system that is capable of acquiring data from a plurality of wireless passive sensors positioned closely within a subject and in sensors placed in multiple, optionally ambulatory, subjects.

In addition to limiting the effects of interference between wireless sensors operating in close proximity to one another, wireless sensing systems that continuously monitor subjects tend to have limited battery life. Although continuous monitoring may be necessary for certain patients with severe medical conditions, most patients don't require continuous monitoring. As a result, battery life can be significantly prolonged by periodic, rather than continuous, data collection and communication.

The presently disclosed ambulatory sensing system and associated methods are directed to overcoming one or more of the problems set forth above and/or other problems in the art.

### SUMMARY

According to one aspect, the present disclosure is directed to a method for monitoring data in a clinical environment. The method may comprise receiving information indicative of a number of ambulatory sensing systems in a measurement environment comprising a plurality of ambulatory sensing systems. The method may also comprise establishing a communication scheme associated with the measurement environment based on the number of ambulatory sensing systems in the measurement environment. The method may further comprise providing, to each ambulatory sensing system, information indicative of the established communication scheme. The method may also comprise configuring, in a central data module associated with the measurement environment, parameters for communicating with each respective ambulatory sensing system in accordance with the established control scheme.

In accordance with another aspect, the present disclosure is directed to an ambulatory sensing system, comprising a first passive sensor configured for implantation within a first subject and a second passive sensor configured for implantation within a second subject. The system may also comprise a first transceiver disposed outside of the first subject. The first transceiver may be configured to generate an electromagnetic signal configured to energize at least a portion of the first passive sensor, and measure feedback from the first passive sensor in response to the generated electromagnetic signal. The system may also comprise a second transceiver disposed outside of the second subject. The second transceiver may be configured to generate an electromagnetic signal configured to energize at least a portion of the second passive sensor, and measure feedback from the second passive sensor in response to the generated electromagnetic signal. While the first transceiver transmits the electromagnetic signal, the second transceiver is prevented from transmitting an electromagnetic pulse.

According to another aspect, the present disclosure is directed to a base unit for an ambulatory sensing environment, comprising a memory, a communications module configured for wireless coupling to one or more ambulatory sensing system, and a processing module. The processing module may be configured to receive information indicative of a number of ambulatory sensing systems in a measurement environment comprising a plurality of ambulatory sensing systems. The processing module may also be configured to establish a communication scheme associated with the measurement environment based on the number of ambulatory sensing systems in the measurement environment. The processing module may be further configured to provide, to each ambulatory sensing system, information indicative of the established communication scheme. The processing module may also be adapted to configure, in a central data module associated with the measurement environment, parameters for communicating with each respective ambulatory sensing system in accordance with the established control scheme.

### BRIEF DESCRIPTION OF THE DRAWINGS

FIG. 1 provides a diagrammatic view of an exemplary ambulatory sensing environment, in accordance with the disclosed embodiments;

FIG. 2 is a block diagram of an embodiment of a ambulatory system which can be used in conjunction with certain of the disclosed embodiments;

FIGS. 3A and 3B schematically illustrates an exemplary substantially planar LC resonant circuits, in accordance with certain disclosed embodiments;

FIG. 4 is an exemplary cross-sectional perspective view of the LC resonant circuit of FIG. 3B;

FIG. 5A schematically illustrated two stagger tuned loops;

FIG. 5B illustrates the assembly of two stagger-tuned loops 502, 504 for transmitting the energizing signal to the passive electrical resonant circuit of the assembly and one un-tuned loop 506 for receiving the output signal;

FIG. 6 provides a block diagram illustrating certain exemplary components associated with the processor-based computing device, in which various processing schemes, such as the communication polling system for ambulatory environments, may be implemented;

FIG. 7 provides a flowchart 700 illustrating an exemplary process for limiting communication interference in ambulatory sensing environments, consistent with certain disclosed embodiments; and

FIG. 8 provides a flowchart 800 illustrating another exemplary process for limiting communication interference in ambulatory sensing environments, in accordance with certain disclosed embodiments.

### DETAILED DESCRIPTION

FIG. 1 illustrates an exemplary interrogation system for communicating with the wireless apparatus described above that is positioned within a body. Without limitation, it is contemplated that the system can be used in at least two environments: the operating room during implant and the physician's office during follow-up examinations.

In one exemplary embodiment, the ambulatory measurement environment can comprise a ambulatory sensing module 100, a base unit 102, a display device 104, and an input device 106, such as, for example and without limitation, a keyboard. In one exemplary embodiment, the base unit 102 can include an RF amplifier, a receiver, and signal processing circuitry. In one aspect, an antenna (denoted as 251, 271 of FIG. 2) associated with ambulatory sensing module 100 can be configured to charge the passive electrical resonant circuit of the sensor assembly and then couple output signals from the energized passive electrical resonant circuit of the sensor assembly into the receiver. Schematic details of the exemplary circuitry are illustrated in greater detail in FIG. 2.

The display 104 and the input device 106 can be used in connection with the user interface for the system. In the embodiment illustrated in FIG. 1, the display device and the input device are conventionally connected to the base unit. In this embodiment, the base unit 102 can also provide conventional computing functions. In other embodiments, the base unit can be connected to a conventional computer 102, a laptop 130, or a mobile computing device 110, such as a tablet or smartphone, via a communications link, such as ZIGBEE or conventional wireless LAN. If a separate computer is used, then the display device and the input devices associated with the computer can be used to provide the user interface. In one embodiment, LABVIEW software can be used to provide the user interface, as well as to provide graphics, store and organize data and perform calculations for calibration and normalization. The user interface can record and display patient data and guide a user through surgical and follow-up procedures. In another aspect, printers, displays, or other peripheral devices (not shown) can be operably connected to the base unit and can be used to display or record patient data or other types of information. As will be apparent to those skilled in the art in light of this disclosure other configurations of the system, as well as additional, different, and/or fewer components can be utilized with embodiments of the invention.

In one embodiment, the antenna (e.g., a loop antenna) associated with ambulatory sensing module 100 can be formed from a band of copper. In this aspect, it is contemplated that the ambulatory sensing module 100 comprises switching and filtering circuitry that is enclosed within a shielded box. In this aspect, the ambulatory sensing module 100 can be configured to charge the passive electrical resonant circuit (such as a passive sensor implant 120) of the assembly and then couples signals from the energized passive electrical resonant circuit 120 of the assembly sensor into a receiver. It is contemplated that the antenna and other components associated with ambulatory sensing module 100 can be shielded to attenuate in-band noise and electromagnetic emissions.

In one aspect, the loop antenna can provide isolation between the energizing signal and the output signal, support sampling/reception of the output signal soon after the end of the energizing signal, and minimize switching transients that can result from switching between the energizing and the coupled mode. Depending upon the desired performance of the system the antenna can also provide a relatively wide bandwidth.

In one embodiment, separate loops can be used for transmitting the energizing signal to the passive electrical resonant circuit of the sensor assembly and coupling the output signal from the energized passive electrical resonant circuit 102 of the sensor assembly. Two stagger-tuned loops can be used to transmit the energizing signal and an un-tuned loop with a high input impedance at the receiver can be used to receive the output signal. The term “coupling loop” is used herein to refer to both the loop(s) used to receive the output signal from the energized passive electrical resonant circuit 120 of the sensor assembly (the “assembly coupling loop”), as well as the loop assembly that includes the loop(s) used to transmit the energizing signal to the passive electrical resonant circuit of the sensor assembly (the “energizing loop”) and the sensor assembly coupling loop(s).

The resonant frequencies for the loops are based on the bandwidth of interest. If there are two loops, then the loops can be spaced geometrically. In one exemplary non-limiting aspect, the resonant frequency of the first loop can be about 31 MHz and the resonant frequency of the second loop can be about 36.3 MHz, which corresponds to the pole locations of a second order Butterworth bandpass filter having about −3 dB points at about 30 MHz and about 37.5 MHz. Although FIGS. 5A and 5B illustrate two loops, it is contemplated that other embodiments can use a different number of loops, which provides coverage for a much wider frequency range. In one aspect, the loops can be spaced logarithmically if there are more than two loops.

During the measurement cycle, the assembly coupling loop can be configured to couple the output signal from the energized passive electrical resonant circuit of the sensor assembly, which is relatively weak and dissipates quickly. In one aspect, the voltage provided to the RF receiver ambulatory sensing module 100 depends upon the design of the assembly coupling loop and in particular, the resonant frequency of the loop. Exemplary embodiments of antenna loops are illustrated in greater detail in FIGS. 5A and 5B.

According to one embodiment, passive electrical resonant circuit 120 may be configured for in-vivo implantation in a subject and may be configured as a passive sensor implant device. Passive sensor implant 120 can comprise a passive LC resonant circuit with a varying capacitor. Because the exemplary assembly can be fabricated using passive electrical components and has no active circuitry, it does not require on-board power sources such as batteries, nor does it require leads to connect to external circuitry or power sources. These features create an assembly which is self-contained within the enclosure and lacks physical interconnections that traverse the hermetic enclosure or housing.

Because of the presence of the inductor in the LC resonant circuits described herein, it is possible to couple to the assembly having the LC resonant circuit (120) electromagnetically and to induce a current in the LC resonant circuit via an antenna associated with ambulatory sensing module 100. This characteristic allows for wireless exchange of electromagnetic energy with the assembly and the ability to operate it without the need for an on-board energy source such as a battery. The presently disclosed ambulatory system is configured to provide an in-vivo assessment of certain physiological parameters of a living being (e.g., using passive sensor implant 120) and detect the physiological parameters using an ex-vivo source of RF energy (e.g., using the antenna associated with ambulatory sensing module 100).

In another aspect, the system described herein provides for a system capable of determining the resonant frequency and bandwidth of the passive sensor implant 120 using an impedance approach. In this approach, an excitation signal can be transmitted using an antenna associated with ambulatory sensing module 100 to electromagnetically couple the passive sensor implant 120 to the ambulatory sensing module 100, which modifies the impedance of the coupling loop 120. The measured change in impedance of the coupling loop 120 allows for the determination of the resonant frequency and bandwidth of the passive sensor implant 120 of the assembly.

According to an exemplary embodiment, the ambulatory sensing system described herein provides for a transmit and receive system configured to determine the resonant frequency and bandwidth of the passive sensor implant within a particular assembly. In this process, an excitation signal of white noise or predetermined multiple frequencies can be transmitted from the antenna and the passive sensor implant 120 is electromagnetically coupled to the ambulatory sensing module 100. A current is induced in the passive electrical resonant circuit of the assembly as it absorbs energy from the transmitted excitation signal, which results in the oscillation of the passive electrical circuit at its resonant frequency. A receiving antenna, which can also be electromagnetically coupled to the ambulatory sensing module 100, receives the excitation signal reduced by the energy which was absorbed by the assembly. Thus, the power of the received or output signal experiences a dip or notch at the resonant frequency of the assembly. The resonant frequency and bandwidth can be determined from this notch in the power. In one aspect, the transmit and receive methodology of determining the resonant frequency and bandwidth of a passive electrical resonant circuit of an assembly can include transmitting a multiple frequency signal from a transmitting antenna to electromagnetically couple the passive sensor implant 120 on the sensor assembly to the transmitting antenna in order to induce a current in the passive sensor implant 120 of the assembly. A modified transmitted signal based on the induction of current in the passive electrical circuit is received and processed to determine the resonant frequency and bandwidth.

According to another exemplary embodiment, the base unit determines the resonant frequency of the sensor by adjusting the energizing signal so that the frequency of the energizing signal matches the resonant frequency of the sensor. In this embodiment, two separate processors and two separate coupling loops may be used. In other embodiments, a single processor is used that provides the same functions as the two separate processors. In yet other embodiments, a single loop is used for both energizing and for coupling the sensor energy back to the receiver. As will be apparent to those skilled in the art, other configurations of the base unit are possible that use additional and/or different components.

According to one embodiment, one of a pair of phase lock loops (PLLs) is used to adjust the phase of the energizing signal and is referred to herein as the fast PLL. The other of the pair of PLLs is used to adjust the frequency of the energizing signal and is referred to herein as the slow PLL. The base unit provides two cycles: the calibration cycle and the measurement cycle. In one embodiment, the first cycle is a 10 microsecond energizing period for calibration of the system, which is referred to herein as the calibration cycle, and the second cycle is a 10 microsecond energizing/coupling period for energizing the sensor and coupling a return signal from the sensor, which is referred to herein as the measurement cycle. During the calibration cycle, the system generates a calibration signal for system and environmental phase calibration, and during the measurement cycle the system both sends and listens for a return signal, i.e. the sensor ring down. Alternatively, as those skilled in the art will appreciate, the calibration cycle and the measurement cycle can be implemented in the same pulse repetition period.

The phase of the energizing signal is adjusted during the calibration cycle by the fast PLL, and the frequency of the energizing signal is adjusted during the measurement cycle by the slow PLL. The following description of the operation of the PLLs is presented sequentially for simplicity. However, as those skilled in the art will appreciate, the PLLs actually operate simultaneously.

Initially the frequency of the energizing signal is set to a default value determined by the calibration parameters of the sensor. Each sensor is associated with a number of calibration parameters, such as frequency, offset, and slope. An operator of the system enters the sensor calibration parameters into the system via the user interface and the system determines an initial frequency for the energizing signal based on the particular sensor. Alternatively, the sensor calibration information could be stored on portable storage devices, bar codes, or incorporated within a signal returned from the sensor. The initial phase of the energizing signal is arbitrary.

During the calibration cycle, the calibration signal which enters the receiver is processed through the receive section and the IF section, and is sampled. In one embodiment, the calibration signal is the portion of the energizing signal that leaks into the receiver (referred to herein as the energizing leakage signal). The signal is sampled during the on time of the energizing signal by a sample and hold circuit to determine the phase difference between the signal and local oscillator. In the embodiment where the calibration signal is the portion of the energizing signal that leaks into the receiver, the signal is sampled approximately 100 ns after the beginning of the energizing signal pulse. Since the energizing signal is several orders of magnitude greater than the coupled signal, it is assumed that the phase information associated with the leaked signal is due to the energizing signal and the phase delay is due to the circuit elements in the coupling loop, circuit elements in the receiver, and environmental conditions, such as proximity of reflecting objects.

During the measurement cycle, the energizing signal may be blocked from the receiver during the on time of the energizing signal. During the off time of the energizing signal, the receiver is unblocked and the coupled signal from the sensor (referred to herein as the coupled signal or the sensor signal) is received. The coupled signal is amplified and filtered through the receive section. The signal is down converted and additional amplification and filtering takes place in the IF section.

In other embodiments, group delay or signal amplitude is used to determine the resonant frequency of the sensor. The phase curve of a second order system passes through zero at the resonant frequency. Since the group delay (i.e. the derivative of the phase curve) reaches a maximum at the resonant frequency, the group delay can be used to determine the resonant frequency. Alternatively, the amplitude of the sensor signal can be used to determine the resonant frequency. The sensor acts like a bandpass filter so that the sensor signal reaches a maximum at the resonant frequency.

FIG. 2 illustrates a schematic diagram of an exemplary ambulatory environment in accordance with one embodiment. As illustrated in FIG. 2, the ambulatory environment may include a plurality of ambulatory units 250, 270 (each associated with a respective patient 261, 281) and a base unit 102. Ambulatory units 250, 270 and the base unit 102 can be communicatively connected through a communication link. This disclosure contemplates the communication link is any suitable communication link. For example, a communication link may be implemented by any medium that facilitates data exchange between the ambulatory units 250, 270 and the base unit 102 including, but not limited to, wired, wireless and optical links. For example, the ambulatory units 250, 270 and the base station 102 can be configured to communicate with each other using a wireless communication protocol such as WiFi, BLUETOOTH or ZIGBEE, for example. It should be understood that other standard or proprietary protocols can optionally be used by the ambulatory units 250, 270 and the base station 102.

The ambulatory units 250, 270 may each include a respective antenna or coupling loop 251, 271, an electronics unit 252, 272 (e.g., RF processing unit), a power source 253, 273 (e.g., battery), and a telemetry circuit 254, 274. Optionally, as discussed above, the ambulatory units 250, 270 can be an external measurement system that is worn by the patient. Each of antennas 251, 271 is configured to inductively couple energy to a sensor such as the implantable MEMS-based pressure sensor discussed above, for example, and receive return signals from the sensor. The return signals received at the ambulatory unit 250, 270 via the respective antenna 251, 271 can be processed by the electronics unit 252, 272. For example, each of the electronics units 252, 272 can be configured to determine a pressure measurement from the signals as described in U.S. Pat. No. 7,679,355, entitled “Communicating with an Implanted Wireless Sensor,” which is incorporated by reference herein in its entirety. As discussed below, the processed signal can be transmitted the base unit 102 for storage and/or further processing. In its most basic configuration, each of the electronics units 252, 272 can include a processing unit and memory. The memory can optionally be volatile or non-volatile memory or some combination of the two. The processing unit can be configured to perform the arithmetic and logic operations necessary for operation of the ambulatory units 250, 270. For example, the processing unit can be configured to execute program code encoded in the memory. Additionally, the power sources 253, 273 can provide power to the other components of the ambulatory unit such as antenna 251, 271, the electronics unit, 252, 272 and the telemetry circuit 254, 274. The telemetry circuits 254, 274 allow the corresponding ambulatory unit 250, 270 to communicate with other devices such as the base unit 102, for example, over the communication link. The telemetry circuits 254, 274 can optionally include a transceiver and a microprocessor. Additionally, the transceiver and microprocessor can optionally be the same component. It should be understood that the ambulatory units 250, 270 discussed above is provided only as an example and that the ambulatory units 250, 270 can include more or less features than those discussed above.

According to one embodiment, the base unit 102 may include a telemetry circuit 220, a data processing circuit 210, a network connection 230, and a user interface 240. Optionally, the base unit 102 is a computing device such as desktop computer 104, laptop computer 130, tablet device 110, etc. The telemetry circuit 220 is similar to the telemetry circuit discussed above and is therefore not discuss in further detail below. In its most basic configuration, the data processing unit 210 can include a processing unit and memory. The memory can optionally be volatile or non-volatile memory or some combination of the two. The processing unit can be configured to perform the arithmetic and logic operations necessary for operation of the base unit 102. For example, the processing unit can be configured to execute program code encoded in the memory. Additionally, the network connection 230 allows the base unit 102 to communicate with other devices. For example, the base unit 102 can optionally be connected to a computer network such as a LAN, WAN, MAN, etc. via the network connection 230.

According to one embodiment, the user interface 240 can include one or more input device (e.g., keyboard, touch screen, mouse, etc.) and/or output devices (e.g., display screen, speakers, printers, etc.). The above components are well known in the art and are therefore not discussed in further detail below. It should be understood that the base unit 102 discussed above is provided only as an example and that the base unit 102 can include more or less features than those discussed above.

Optionally, to allow effective monitoring of patients while active (e.g., during exercise), the ambulatory units 250, 270 can be designed to be worn on the patient's body. For example, harnessing methods can be used to fix the ambulatory units 250, 270 properly to the patient. In one example implementation, the electronics unit 252, 272 and battery 253, 273 can be contained within one or more pockets of a garment such as a vest. The electronics unit 252, 272 and the battery 253, 273 can optionally be combined into a single housing or can optionally be housed separately.

One will appreciate that the signal from an implanted passive assembly is relatively weak and is attenuated by the surrounding tissue and the distance between the assembly and the coupling loop. Optimizing the position and angle of the coupling loop relative to the assembly can help maximize the coupling between the assembly and the coupling loop. In one aspect, the coupling loop can be positioned so that a plane defined by the assembly coupling loop is approximately parallel to the inductor within the passive electrical resonant circuit of the assembly and the assembly is approximately centered within the sensor coupling loop. If the coupling loop is not positioned in this manner relative to the inductor, then the strength of the coupled signal is reduced by the cosine of the angle between the sensor coupling loop and the inductor of the resonant circuit.

To provide for the adequate signal integrity, each antenna 251, 271 can be positioned in a fixed position with respect to the sensor (e.g., the implanted MEMS-based sensor). Positioning of the antenna 251, 271 can be flexible to provide for optimal coupling, and therefore, the means of fastening the antenna 251, 271 can provide options for adjusting positioning. These options include movement of antenna 251, 271 physically to another location and/or adjusting/tightening straps to move the relative location of a permanently fixed antenna 251, 271 to a more optimal position. Optionally, the antenna 251, 271 can be sewn in place within a garment such as a vest, or the antenna 251, 271 can be fixed by some means of a temporary fabric fastener such as hook and loop (e.g., VELCRO). Optionally, in the Heart Failure applications, the antenna 251, 271 can be fixed on the patient's back, for instance, over the patient's shoulder blade and can be communicatively connected to the electronics unit 252, 272 with a cable. Alternatively, the antenna 251, 271 can optionally communicate signals received by the antenna 251, 271, for example, over a short range, directly to the base unit 102. In other words, the base unit 102 can be configured to determine a pressure measurement from the signals as discussed above. As discussed above, the ambulatory units 250, 270 can communicate with the base unit 102 over the communication link, which can be a wireless communication link, for example. The ambulatory units 250, 270 can optionally be worn by the patient who is located remotely from the base unit 102. The ambulatory units 250, 270 can communicate data including, but not limited to, data read from the sensors (e.g., the implanted MEMS-based sensors), diagnostic data from the ambulatory units 250, 270 and barometric pressure. The diagnostic data can include status information such battery status, temperature, etc. This disclosure contemplates that the base unit 102 can communicate with a plurality of ambulatory units at the same time.

According to one embodiment, a plurality of patients, each having an ambulatory unit, can be monitored at the same time. As will be explained in greater detail below, transmission/reception of data between the base unit 102 and a plurality of ambulatory units can be synchronized to prevent interference. In one implementation, the base unit 102 can control synchronization. For example, each of rgw ambulatory units 250, 270 can be associated with a unique identifier, and the base unit 102 can poll a specific ambulatory unit using its unique identifier to initiate communication. In other words, the base unit 102 can be configured to send a message to an ambulatory unit, and the ambulatory unit can “wake up” and communicate with the base unit 102 upon receipt.

Alternatively, the plurality of ambulatory units can control synchronization. For example, each of the ambulatory units can be configured to monitor communications between the other ambulatory units and the base unit 102. The base unit 102 can have a predetermined duty cycle for transmission and reception (e.g., a predetermined transmit-read cycle). The predetermined duty cycle can optionally be 10 microseconds. The base unit 102 can therefore be repeatedly available to receive communications at specified times. It should be understood that the predetermined duty cycle can be more or less than 10 microseconds. The plurality of ambulatory units 250, 270 can synchronize their respective wake periods (e.g., communication periods) into a predetermined order. Alternatively or additionally, the plurality of ambulatory units 250, 270 can agree upon a synchronization order through a networking algorithm. This can optionally be achieved through direct communication from the base unit 102 (e.g., a star network) or through ambulatory unit to ambulatory unit communication in a repeater pattern (e.g., a mesh network).

Optionally, the ambulatory units 250, 270 can be configured to use one or more duty cycle sequences (e.g., ON/OFF, wake/sleep, etc.) to reduce power requirements. By reducing the power drawn by the ambulatory units 250, 270, the useful battery life can be extended. In other words, the ambulatory units 250, 270 can operate for a longer period of time without re-charging its power source 253, 273. The ambulatory units 250, 270 can be configured to have a “wake” mode in which the ambulatory units 250, 270 are capable of communicating and/or are communicating with the base unit 102 and a “sleep” mode where their power requirements are reduced. Optionally, in the sleep mode, the ambulatory units 250, 270 can operate in a lower power state such that power is supplied only to components necessary to “wake-up” the ambulatory unit 250, 270. For example, the telemetry circuit 254 can optionally be configured to send a message to the ambulatory units 250, 270 to initiate the wake mode. The wake mode-sleep mode cycle can define the duty cycle sequence.

Alternatively or additionally, the ambulatory units 250, 270 can be configured to implement an intra-sample duty cycle, which can be provided to decrease power requirements of the ambulatory units 250, 270. The intra-sample duty cycle can be reduced to minimize transmission power of the ambulatory unit 250, 270. The intra-sample duty cycle can optionally be the amount of time that each of ambulatory units 250, 270 communicates with/transmits data to the base unit 102 during its wake mode. The intra-sample duty cycle can be optimized for the Q of the sensor (e.g., the implanted MEMS-based sensor) and/or the apparent power of the return signals received from the sensor. For example, the ambulatory units 250, 270 can optionally optimize the amount of time they communicate with/transmit to the base unit 102. The ambulatory units 250, 270 can optionally be configured to monitor the amplitude of the return signals received from the sensor and reduce the amount of transmission time (e.g., on-time) while the amplitude of the return signal is below a specified threshold. The specified threshold can correspond to a minimum signal to noise ratio. The ambulatory units 250, 270 can be configured to reduce transmission time when the signal to noise ratio of the return signal is low because useful data might not be recovered due to the low return signal strength. Additionally, the ambulatory units 250, 270 can be configured to monitor the amplitude of the return signals received from the sensor and increase the amount of transmission time (e.g., on-time) while the amplitude of the return signal is above a specified threshold.

The base unit 102 can initiate the reading process by accepting the parameter information specific to the patient's sensor, reading parameters (such as length of readings, sample rates, and frequency ranges, etc.) and patient demographic information. The base unit 102 can then communicate the set-up parameters to the ambulatory units 250, 270, which initiate the reading process. The base unit 102 can also be configured to display the signal strength from the coupled sensor, for example, on the user interface 240. The base unit 102 can optionally be configured to initiate a frequency scan to locate the sensor within a prescribed frequency range if the signal strength is weak. Additionally, the base unit 102 can also be configured to calculate the pressure measured by the sensor given a calibrated pressure sensor during the measurements. The base unit 102 can also be configured to handle data acquisition requests by acquiring several seconds of measured pressure data, which can include systolic, diastolic, pulse, cardiac output and/or mean pressure values for the acquisition period. The base unit 102 can also be configured to store data to memory. Alternatively or additionally, the base unit 102 can also be configured to communicate data such as the wave form and cardiac output, systolic, diastolic, pulse and mean pressure data to a remote computer, for example, a data server of a monitoring system. As discussed above, the base unit 102 is network connectable. Optionally, the base unit 102 can be configured to perform the above functions in real-time. Optionally, the base unit 102 can be configured to perform the above functions on a single patient or multiple patients either simultaneously or in succession.

The data from the readings can optionally be accessed and/or analyzed remotely, for instance, through an application accessed through a network (e.g., the Internet) or through an application resident on the base unit 102. The application can allow a user to access data from individual readings or access historical data from individual patients. The application provides the ability to track trends of physiological measurements and associates those trends with events, activity information, and other physiological data and medication changes. This data can then be used by a clinician to more effectively treat the patient's disease state.

According to one embodiment, although the base unit 102 is illustrated as being directly communicatively coupled to each of ambulatory units 250, 270, it is contemplated that base unit 102 may be capable of direct communication with fewer than all of the ambulatory units 250, 270 at any given time. It is contemplated that if base unit 102 cannot directly communicate with one or more ambulatory units 250, 270, it may indirectly communicate with it through one or more other ambulatory units that it does have direct communication with. In this manner, the ambulatory sensing environment may be configured as a mesh network, wherein each of the ambulatory units may be configured as a bridge node between other ambulatory units and base unit 102.

FIGS. 3A and 3B illustrate alternate passive sensor designs that may be implemented in an ambulatory sensing system consistent with the disclosed embodiments. FIG. 3A is a top, partial cross-section of a schematic representation of sensor 200 where a wire spiral inductor coil 304 is positioned in planar fashion in a substrate 306. Optionally sensor 300 may have recesses 308, each with a hole 310, to receive a tether wire (not shown here) for delivery of the device into a human patient, as described below. In the embodiment shown in FIG. 3B, a wire 312 connects coil 304 to a capacitor plate 314 positioned within coil 304.

FIG. 4 is a slightly oblique cross-section across its width of the embodiment shown in FIG. 3B where it can be seen that sensor 300 is comprised of a lower substrate 420 and an upper substrate 422. Lower substrate 420 and upper substrate 422 are constructed from a suitable material, such as glass, fused silica, sapphire, quartz, or silicon. Fused silica is the preferred material of construction. Lower substrate 420 has on its upper surface 424 an induction coil 426 and capacitive plate 436, and upper substrate 422 has a recess 428 with a surface 430 having an induction coil 432 and capacitive plate 438 thereon. The top surface of upper substrate 422 forms a membrane 434 capable of mechanically responding to changes in a patient's physical property, such as pressure. The end 437 of sensor 300 has a notch or recess 438.

FIGS. 5A and 5B illustrate exemplary coupling loops 100 that may be implemented in accordance with the disclosed embodiments. As illustrated in FIGS. 5A and 5B, the assembly of two stagger-tuned loops 502, 504 for transmitting the energizing signal to the passive electrical resonant circuit of the assembly and one un-tuned loop 506 for receiving the output signal. In this aspect, the loops are parallel to one another with the un-tuned loop inside the stagger-tuned loops. Placing the loop used to receive the output signal inside of the loops used to transmit the energizing signal helps to shield the output signal from environmental interferences. In one embodiment, the loops can be positioned within a housing.

In yet another aspect, orientation features can be provided for positioning the coupling loop relative to at least the first assembly to maximize the coupling between the first assembly and the coupling loop. In one aspect, the orientation features can facilitate the placement of the respective assemblies during implantation and the placement of the coupling loop during follow-up examinations. In one aspect, the respective assemblies and the coupling loop can include orientation features that are visible using conventional medical imaging technology. In exemplary aspects, the orientation features on the assemblies can include radiopaque markings and the orientation features on the coupling loop can include a pattern in the ribbing of the housing for the loop.

In one exemplary aspect, to facilitate the proper coupling of the system, the assembly, the assembly housing, and/or the implant can include orientation features, which are visible using a medical imaging technology, such as fluoroscopy, to facilitate the placement of the assemblies during implantation and the coupling loop during follow-up examinations. To position the coupling loop relative to the assembly, the coupling loop is moved or adjusted until a predetermined pattern appears. In one aspect, the orientation features on the coupling loop can be implemented as a pattern in the ribbing of the housing for the loop, which aids in positioning the coupling loop relative to the assembly of the implant. In one aspect, the housing includes an essentially circular section that can be smaller than the diameter of section. When assembled, the sensor coupling and energizing loops are positioned within the ring-shaped section. The orientation features are located in the circular section.

As illustrated in FIG. 6, processing system 610 may include one or more hardware and/or software components configured to execute software programs, such as software for processing and communicating data associated with an ambulatory sensing system in a clinical environment. According to one embodiment, processing system 610 may include one or more hardware components such as, for example, a central processing unit (CPU) or microprocessor 611, a random access memory (RAM) module 612, a read-only memory (ROM) module 613, a memory or data storage module 614, a database 615, one or more input/output (I/O) devices 616, and an interface 617. Alternatively and/or additionally, processing system 610 may include one or more software media components such as, for example, a computer-readable medium including computer-executable instructions for performing methods consistent with certain disclosed embodiments. It is contemplated that one or more of the hardware components listed above may be implemented using software. For example, storage 614 may include a software partition associated with one or more other hardware components of processing system 610. Processing system 610 may include additional, fewer, and/or different components than those listed above. It is understood that the components listed above are exemplary only and not intended to be limiting.

CPU 611 may include one or more processors, each configured to execute instructions and process data to perform one or more functions associated with processing system 610. As illustrated in FIG. 6, CPU 611 may be communicatively coupled to RAM 612, ROM 613, storage 614, database 615, I/O devices 616, and interface 617. CPU 611 may be configured to execute sequences of computer program instructions to perform various processes, which will be described in detail below. The computer program instructions may be loaded into RAM 612 for execution by CPU 611.

RAM 612 and ROM 613 may each include one or more devices for storing information associated with an operation of processing system 610 and/or CPU 611. For example, ROM 613 may include a memory device configured to access and store information associated with processing system 610, including information for identifying, initializing, and monitoring the operation of one or more components and subsystems of processing system 610. RAM 612 may include a memory device for storing data associated with one or more operations of CPU 611. For example, ROM 613 may load instructions into RAM 612 for execution by CPU 611.

Storage 614 may include any type of mass storage device configured to store information that CPU 611 may need to perform processes consistent with the disclosed embodiments. For example, storage 614 may include one or more magnetic and/or optical disk devices, such as hard drives, CD-ROMs, DVD-ROMs, or any other type of mass media device. Alternatively or additionally, storage 614 may include flash memory mass media storage or other semiconductor-based storage medium.

Database 615 may include one or more software and/or hardware components that cooperate to store, organize, sort, filter, and/or arrange data used by processing system 610 and/or CPU 611. For example, database 615 may include historical data such as, for example, patient physiological data. CPU 611 may access the information stored in database 615 to provide a user with the ability to track the condition of the patient.

I/O devices 616 may include one or more components configured to communicate information with a component or user associated with ambulatory sensing environment. For example, I/O devices 616 may include a console with an integrated keyboard and mouse to allow a user to input parameters associated with ambulatory sensing environment. According to one embodiment, I/O devices 616 may be configured to receive one or more add or remove ambulatory sensing systems into the ambulatory sensing environment as additional patients are added to the clinical environment. I/O devices 616 may also include a display including a graphical user interface (GUI) for providing a network management console for health care professional to monitor the condition of patients in the environment. I/O devices 616 may also include peripheral devices such as, for example, a printer for printing information associated with network nodes 130, a user-accessible disk drive (e.g., a USB port, a floppy, CD-ROM, or DVD-ROM drive, etc.) to allow a user to input data stored on a portable media device, a microphone, a speaker system, or any other suitable type of interface device. I/O devices may be configured to output network analysis results and traffic characteristics.

Interface 617 may include one or more components configured to transmit and receive data via a communication network, such as the Internet, a local area network, a workstation peer-to-peer network, a direct link network, a wireless network, or any other suitable communication platform. For example, interface 617 may include one or more modulators, demodulators, multiplexers, demultiplexers, network communication devices, wireless devices, antennas, modems, and any other type of device configured to enable data communication via a communication network. According to one embodiment, interface 617 may be coupled to or include wireless communication devices, such as a module or modules configured to transmit information wirelessly using Wi-Fi or Bluetooth wireless protocols.

Processes and methods consistent with the disclosed embodiments may provide system for ensuring that signals associated with the individual sensing units in the presently described ambulatory sensing environment do not interfere with one another. FIGS. 7 and 8 illustrate exemplary flowcharts 700 and 800 describing exemplary processes by which polling in ambulatory sensing environment may be implemented.

As illustrated in FIG. 7, the process for communicating with one or more ambulatory sensing units may commence upon receipt of configuration information indicative of the ambulatory environment (Block 710). For example, a data processing system 210 associated with the base station may receive registration information from each of ambulatory sensing systems 250, 270 that are to be operated in a clinical environment. In response, the base station may determine the number of ambulatory sensing systems that are to be operated in the environment in order to establish a communication scheme for the measurement environment.

Once the configuration information has been received, a communication scheme associated with the measurement environment may be established based, at least in part, on the number of ambulatory sensing systems that are to be operated in the environment (Block 720). According to one exemplary embodiment, the base station processor 210 may assign each of the ambulatory sensing systems a unique ID code and corresponding communication time slot that provides the respective ambulatory sensing system with the exclusive ability to transmit and receive measurement data from the subject. For example, if there are 16 ambulatory sensing system operating in a clinical environment, the base station may assign each unit it own exclusive time slot within a communication cycle. Each unit will transmit and receive measurement data only during the specific allotted time slot, remaining “silent” during all other times, so as not to interfere with other sensing units operating in the environment.

Alternatively or additionally, the communication scheme may include a queue system, whereby each of the ambulatory sensing systems registers for a particular time slot in queue, based on certain patient-specific criteria. For example, if the ambulatory sensing system is being worn by a patient with a critical health emergency, the corresponding sensing system associated with such patient may be given priority over one or more other sensing systems (presumably corresponding with patients having less severe health conditions). In certain embodiments, even when predetermined time slots are assigned, priority conditions (such as health emergencies) may be allowed to move up or down in the time slot queue. As such, if one of the ambulatory sensing systems detects a critical change in pulse rate, for example, such sensing system may request an interruption in the normally-assigned time slot configuration in order to deliver the critical measurement data back to the base station.

Once the communication scheme has been established, information indicative of the communication scheme may be provided to each of the ambulatory sensing system (Block 730). Such information may include, for example, the time slot schedule assignment for each of the communication, the priority assignments for each of the ambulatory systems, and any other data that may be used to configure the communication scheme by the individual ambulatory sensing systems. Once the communication scheme information has been delivered to each ambulatory sensing system, the central module (i.e., base station) may communicate with the ambulatory sensing environment (and its constituent sensing systems) in accordance with the established communication scheme (Block 740).

FIG. 8 illustrates a flowchart 800 that outlines an alternative process for establishing communications in an ambulatory sensing environment. As illustrated in FIG. 8, the process may commence by the establishment of a communication queue for sensing systems in the measurement environment (Block 810). As explained with respect to flowchart 700 of FIG. 7, the queue process may be based on predetermined time slot assignments associated with each of the ambulatory sensing systems, a priority-based system that uses patient-specific health information to establish a priority associated with each of the ambulatory sensing units, a “ping-and-listen” approach whereby the control system sends transmit and receive commands to each of the sensing units, or a hybrid system that utilizes one or more of these schemes.

Once the communication queue has been established, the queue assignment may be transmitted or uploaded to each of the ambulatory sensing units in the measurement environment (Block 820). The measurement environment may begin operating based on the communication queue, unless or until a queue interrupt request is received (Block 830: Yes). For example, if one of the ambulatory sensing units measures a critical health condition associated with the patient, it may generate an interrupt request to allow for urgent delivery of the measurement data to the base station controller.

If an interrupt request is received, the base station may provide a poll signal to the ambulatory system that transmitted the request (Block 850) to receive the measurement data from the polled ambulatory sensing system (Block 860). If no queue interrupt request is received (Block 830: No), the base station may send the poll signal to the next ambulatory sensing system in the queue (Block 840) to receive the measurement data from the polled ambulatory sensing system (Block 860). It is contemplated that, in certain embodiments, in addition to the “poll” signal that is sent to one ambulatory sensing system, a corresponding signal may be simultaneously sent to the other ambulatory sensing systems, causing the other sensing units to remain “RF” silent, so as to avoid unnecessary interference during the data monitoring process.

It will be apparent to those skilled in the art that various modifications and variations can be made to the disclosed systems and methods for effectively and remotely monitoring physiological parameters of a plurality of mobile subjects. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

What is claimed is:

1. A method for monitoring data in a clinical environment, comprising:
receiving information indicative of a number of ambulatory sensing systems in a measurement environment comprising a plurality of ambulatory sensing systems;
establishing a communication scheme associated with the measurement environment based on the number of ambulatory sensing systems in the measurement environment;
providing, to each ambulatory sensing system, information indicative of the established communication scheme; and
configuring, in a central data module associated with the measurement environment, parameters for communicating with each respective ambulatory sensing system in accordance with the established control scheme.

2. The method of claim 1, wherein establishing the communication scheme associated with the measurement environment comprises identifying individual ones of the plurality of ambulatory sensing systems, each of the ambulatory sensing system comprising a respective passive sensor implant and a corresponding wearable transceiver unit configured to transmit an electromagnetic pulse and responsively measuring feedback from the corresponding passive sensor.

3. The method of claim 2, wherein providing information indicative of the established communication scheme includes providing information for:
causing a first of the wearable transceiver units of the plurality of ambulatory sensing systems to transmit an electromagnetic pulse and responsively measure feedback from the corresponding passive sensor; and
preventing the other of the wearable transceiver units of the plurality of ambulatory sensing system from transmitting an electromagnetic pulse while the first of the wearable transceiver units transmits.

4. The method of claim 3, wherein providing the information for causing the first of a wearable transceiver units to transmit and preventing the other of the wearable transceiver units from transmitting includes:
establishing a queue that assigns a dedicated time period during which each respective wearable transceiver unit is authorized transmit an electromagnetic pulse and responsively measure feedback from the corresponding passive sensor; and
transmitting the established queue to each of the wearable transceiver unit.

5. The method of claim 4, further comprising:
receiving a queue interrupt request generated by one of the wearable transceiver units; and
modifying the queue by assigning the wearable transceiver unit that generated the queue interrupt request into a next available position in the queue.

6. The method of claim 4, wherein establishing the queue includes establishing an order of priority associated with the wearable transceiver units, wherein the order of priority is based on a severity of a medical condition associated with a patient wearing the wearable transceiver unit.

7. The method of claim 1, wherein configuring parameters for communication with each respective ambulatory sensing system includes assigning a respective code to each respective ambulatory sensing system, the respective code for uniquely identifying data received from the respective ambulatory sensing system.

8. The method of claim 1, wherein providing, to each ambulatory sensing system, information indicative of the established communication scheme includes providing the information indicative of the established communication scheme to a first ambulatory sensing system, which responsively forwards the information indicative of the established communication scheme to a second ambulatory sensing system.

9. An ambulatory sensing system, comprising:
a first passive sensor configured for implantation within a first subject;
a second passive sensor configured for implantation within a second subject;
a first transceiver disposed outside of the first subject and configured to:
generate an electromagnetic signal configured to energize at least a portion of the first passive sensor;
measure feedback from the first passive sensor in response to the generated electromagnetic signal; and
a second transceiver disposed outside of the second subject and configured to:
generate an electromagnetic signal configured to energize at least a portion of the second passive sensor;
measure feedback from the second passive sensor in response to the generated electromagnetic signal;
wherein while the first transceiver transmits the electromagnetic signal, the second transceiver is prevented from transmitting an electromagnetic pulse.

10. The system of claim 9, wherein the first and second transceivers are disposed in respective wearable garments that are configured to position the first and second transceivers proximate respective first and second passive sensors.

11. The system of claim 9, further comprising a central data module, communicatively coupled to each of the first and second transceivers and configured to:
cause the first transceiver to transmit an electromagnetic pulse and responsively measure feedback from the corresponding passive sensor; and
prevent the second transceiver from transmitting an electromagnetic pulse while the first of the wearable transceiver units transmits.

12. The system of claim 11, wherein the central data module is further configured to:
establish a queue that assigns a respective time period during which each of first and second transceivers is authorized transmit an electromagnetic pulse and responsively measure feedback from the corresponding passive sensor; and
transmit the established queue to each of the first and second transceivers.

13. The system of claim 12, wherein the central data module is further configured to:
receive a queue interrupt request generated by one of the first or second transceivers; and
modify the queue by assigning the transceiver that generated the queue interrupt request into a next available position in the queue.

14. The system of claim 12, wherein the central data module is configured to establish the queue by establishing an order of priority associated with the first and second transceivers, wherein the order of priority is based on a severity of a medical condition associated with a respective patient wearing the first or second transceiver.

15. A base unit for an ambulatory sensing environment, comprising:
a memory;
a communications module configured for wireless coupling to one or more ambulatory sensing system; and
a processing module, configured to:
receive information indicative of a number of ambulatory sensing systems in a measurement environment comprising a plurality of ambulatory sensing systems;
establish a communication scheme associated with the measurement environment based on the number of ambulatory sensing systems in the measurement environment;
provide, to each ambulatory sensing system, information indicative of the established communication scheme; and
configure, in a central data module associated with the measurement environment, parameters for communicating with each respective ambulatory sensing system in accordance with the established control scheme.

16. The base unit of claim 15, wherein establishing the communication scheme associated with the measurement environment comprises identifying individual ones of the plurality of ambulatory sensing systems, each of the ambulatory sensing system comprising a respective passive sensor implant and a corresponding wearable transceiver unit configured to transmit an electromagnetic pulse and responsively measuring feedback from the corresponding passive sensor.

17. The base unit of claim 16, wherein providing information indicative of the established communication scheme includes providing information for:
causing a first of the wearable transceiver units of the plurality of ambulatory sensing systems to transmit an electromagnetic pulse and responsively measure feedback from the corresponding passive sensor; and
preventing the other of the wearable transceiver units of the plurality of ambulatory sensing system from transmitting an electromagnetic pulse while the first of the wearable transceiver units transmits.

18. The base unit of claim 17, wherein providing the information for causing the first of a wearable transceiver units to transmit and preventing the other of the wearable transceiver units from transmitting includes:
establishing a queue that assigns a dedicated time period during which each respective wearable transceiver unit is authorized transmit an electromagnetic pulse and responsively measure feedback from the corresponding passive sensor; and
transmitting the established queue to each of the wearable transceiver unit.

19. The base unit of claim 18, wherein the processor is further configured to:
receive a queue interrupt request generated by one of the wearable transceiver units; and
modify the queue by assigning the wearable transceiver unit that generated the queue interrupt request into a next available position in the queue.

20. The base unit of claim 18, wherein establishing the queue includes establishing an order of priority associated with the wearable transceiver units, wherein the order of priority is based on a severity of a medical condition associated with a patient wearing the wearable transceiver unit.

21. The base unit of claim 15, wherein configuring parameters for communication with each respective ambulatory sensing system includes assigning a respective code to each respective ambulatory sensing system, the respective code for uniquely identifying data received from the respective ambulatory sensing system.
